(19) United States
(12) Patent Application Publication
(10) Pub. No.: US 2008/0008771 A1
(43) Pub. Date: Jan. 10, 2008

(54) Nitric Oxide Dioxygenase Inhibitors

(75) Inventor: Paul R. Gardner, Dayton, OH (US)

Correspondence Address: WOOD, HERRON & EVANS, LLP, 2700 CAREW TOWER, 441 VINE STREET, CINCINNATI, OH, 45202, US

(73) Assignee: CHILDREN'S HOSPITAL MEDICAL CENTER, Cincinnati, OH (US)

(21) Appl. No.: 11/569,620

(22) PCT Filed: May 26, 2005
(86) PCT No.: PCT/US05/18464

§ 371 Date: Jun. 20, 2007

Related U.S. Application Data
(60) Provisional application No. 60/574,807, filed on May 27, 2004.

(51) Int. Cl. A61K 31/4164; A61K 31/105; A61K 31/496; A61P 31/00; A61P 35/00; A61K 33/00; A61K 31/352

(52) U.S. Cl. 424/699; 424/718; 514/254.07; 514/396; 514/398; 514/399; 514/456; 514/707

(57) ABSTRACT

A method and composition to inhibit the enzyme nitric oxide dioxygenase (NOD) and therefore accumulate nitric oxide (NO) in cells or tissues. By preventing NO removal, the inhibitors may effect cellular signaling, modulate vasotension, enhance O₂ delivery to tissues, and provide antibiotic and/or antineoplastic effects. Inhibitors include compounds that bind to the iron in the heme portion of NOD, and include allicin and azoles.

### RELATED APPLICATION

This application claims priority from U.S. Provisional Application Ser. No. 60/574,807 filed May 27, 2004.

The U.S. Government has a paid-up license in this invention and the right in limited circumstances to require the patent owner to license others on reasonable terms as provided for by the terms of Grant No. GM65090 awarded by the National Institutes of Health.

### FIELD OF THE INVENTION

The invention is directed to inhibitors of nitric oxide dioxygenase and their uses.

### BACKGROUND

Nitric oxide dioxygenases (NOD) catalyze the reaction NO+O₂+e⁻→NO₃⁻. They therefore provide oxidant and free radical defense mechanisms by detoxifying nitric oxide (NO). NO is a radical that builds up to toxic amounts when induced by responses to infections, foreign bodies, or tissue injury. At low levels, NO acts as a signal and controls diverse physiological processes including vasotension and O₂ delivery to tissues. NOD protects diverse cells and organisms from NO poisoning, growth inhibition and killing. NOD also modulates NO signaling pathways controlling vasorelaxation.

The structure and enzymatic function of NOD from Escherichia coli, a flavohemoglobin-type NOD, has been reported (Gardner et al., Proc. Natl. Acad. Sci. USA 95, 13089 (1998) which is expressly incorporated by reference herein in its entirety). The reaction steps for flavohemoglobin-catalyzed NO dioxygenation incorporate the NADPH, FAD, and O₂ dependence, as well as other features, of the mammalian hemoglobin. Mammalian NOD is a microsomal cytochrome P450 oxidoreductase (EC 1.6.2.4)-driven heme-dependent enzyme (Hallstrom et al. Free Radic. Biol. Med. 37(2) (2004)), which is expressly incorporated by reference herein in its entirety).

Uses for NOD are thus desirable.

### SUMMARY OF THE INVENTION

(Flavo)hemoglobins dioxygenate nitric oxide (NO), which is toxic to cells. This reaction is catalyzed by nitric oxide dioxygenase (NOD) and forms nitrate, which protects microbes from NO-mediated growth inhibition and killing in vitro and in infections. Inhibiting NOD allows NO to accumulate and exert its toxic effect. Inhibitors of microbial NOD exert an antimicrobial effect because the inhibitors permit toxic concentrations of NO produced by immune cells, or otherwise delivered, to accumulate in microbial cells. Inhibitors of mammalian NOD exert anti-tumor effects because the inhibitors permit toxic concentrations of NO produced by immune cells, or otherwise delivered, to accumulate in tumor cells and tissues. Inhibitors of mammalian NOD elicit vasorelaxant effects by, for example, increasing the low non-toxic nanomolar NO levels normally modulating vasotension.

Heme-binding antimicrobial imidazoles inhibited NOD activity in vitro, formed a ligand with the catalytic heme iron in NOD, and inhibited NOD function within microbes. Each of miconazole, econazole, clotrimazole and ketoconazole inhibited NOD from Escherichia coli, Alcaligenes eutrophus, and Saccharomyces cerevisiae, with miconazole being the most effective imidazole tested. Miconazole acted non-competitively with respect to O₂ and thus did not compete with O₂ for the ferrous heme, but bound ferric heme and inhibited both hydride transfer from NADH to FAD and electron transfer to the ferric heme. Miconazole inhibited NOD activity within S. cerevisiae and E. coli, and increased the sensitivity of S. cerevisiae to NO-mediated growth inhibition. Thus, azoles and other compounds that bind to heme are able to inhibit NOD with various effects.

One embodiment of the invention is a biocompatible composition of a NOD inhibitor in an amount sufficient to increase the intracellular NO to exert an antimicrobial, antineoplastic, and/or vasorelaxant effect. The inhibitor may be to mammalian or microbial NOD, and may include an azole, allicin, quercetin, carbon monoxide, or cyanide. The composition, for example, an antibacterial, may be formulated for topical administration as a cream, lotion, gel, etc., or for parenteral or enteral delivery.

Another embodiment of the invention is an antimicrobial composition that contains an inhibitor of microbial NOD in an amount sufficient to accumulate a toxic concentration of NO in the microbe to exert an antimicrobial effect. The composition may also contain a peroxide such as hydrogen peroxide or an organic peroxide, hypochlorous acid, and/or lysozyme.

Another embodiment of the invention is an antimicrobial composition containing a subtoxic amount of NO and an amount of an azole, such as miconazole, econazole, metronidazole, ketoconazole, and/or clotrimazole, sufficient to synergistically mediate NO-induced microbial toxicity.

Another embodiment of the invention is a composition containing at least one heme-binding compound in an amount effective to inhibit NOD. The heme-binding compound may be an azole. The heme-binding compound may bind to the distal heme pocket of NOD at a conserved hydrophobic region. The compound may inhibit either mammalian or microbial NOD.

Another embodiment of the invention is a method of reducing microbial growth and activity by trapping a Fe³⁺ intermediate in NOD catalysis of nitric oxide to nitrate, and thereby exerting a microbicidal effect by reducing NO detoxification. An azole may trap the Fe³⁺ intermediate.

Another embodiment of the invention is a method of reducing microbial growth and activity by accumulating an amount of NO that is toxic to a microbe, such as bacteria, by providing an azole and thus inhibiting microbial NOD and NO detoxification.

Another embodiment of the invention is a method of reducing microbial growth and activity by inhibiting NOD-mediated detoxification of NO to nitrate in a microbial cell by providing at least one azole in an inhibitory concentration. In specific embodiments, the inhibitor concentration may range from about 1 nM to about 100 μM.

Another embodiment of the invention is a method to decrease microbial antibiotic resistance by providing an amount of an azole sufficient to inhibit NOD to provide an antimicrobial effect. A subtoxic amount of NO is provided with an amount of an azole sufficient to synergistically effect NO toxicity.

Another embodiment of the invention is a method of inhibiting microbial growth and activity by providing to a microbe an azole in an amount sufficient to ligand with heme in microbial NOD and result in a toxic accumulation of NO to inhibit microbial growth and activity.

Another embodiment of the invention is a method of inhibiting microbial growth and activity by providing an azole inhibitor of NOD that is non-competitive with oxygen and NO in inhibiting NOD catalysis.

Another embodiment of the invention is a method for inhibiting microbial NOD by providing at least one of miconazole, econazole, clotrimazole, ketoconazole, or metronidazole to a organism under conditions sufficient to inhibit microbial NOD.

Another embodiment of the invention is a method of enhancing NO toxicity by providing NO and an inhibitor of NOD under conditions sufficient to reduce NOD-catalyzed detoxification of toxic NO to nitrate.

Another embodiment of the invention is a method of modulating therapy in a patient by providing at least one inhibitor of mammalian NOD in an amount sufficient to accumulate a concentration of NO to modulate an antineoplastic effect or a vasorelaxant effect. This may be in response to a steady state oxygen concentration in the tissue. The inhibitor, such as an azole, allicin, quercetin, carbon monoxide, or cyanide, may increase NO signaling.

These and other advantages will be apparent in light of the following figures and detailed description.

### BRIEF DESCRIPTION OF THE DRAWINGS

FIG. 1 shows imidazole structures (A) miconazole, (B) econazole, (C) clotrimazole, and (D) ketoconazole.

FIG. 2 shows imidazole inhibition of E. coli nitric oxide dioxygenase (NOD).

FIGS. 3A and 3B demonstrate non-competitive inhibition of NOD by miconazole with respect to O₂ (FIG. 3A) and nitric oxide (NO) (FIG. 3B).

FIGS. 4A and 4B are spectra of oxidized (FIG. 4A) and reduced (FIG. 4B) flavohemoglobin and flavohemoglobin-miconazole complexes.

FIGS. 5A and 5B are spectra of flavohemoglobin (Fe3⁺) in the absence (FIG. 5A) or presence (FIG. 5B) of miconazole.

FIGS. 6A and 6B show miconazole inhibition of heme reduction (FIG. 6A) and flavin reduction (FIG. 6B).

FIG. 7 shows a mechanism for imidazole inhibition.

FIGS. 8A and 8B show miconazole inhibition of NO consumption (FIG. 8A) and the time dependence of NO consumption (FIG. 8B).

FIGS. 9A and 9B show synergistic inhibition of growth in parent (FIG. 9A) and flavohemoglobin deficient mutant strains of S. cerevisiae by miconazole and NO (FIG. 9B).

FIG. 10 shows traces of NO metabolism by intact and digitonin-permeabilized Caco-2 cells and the NADPH dependence.

FIGS. 11A and 11B show cyanide and carbon monoxide (CO) sensitivity of cellular NO metabolism.

FIGS. 12A, 12B, and 12C show NO, NADPH, and O2 dependence, respectively, on microsomal NO metabolism.

FIGS. 13A, 13B, and 13C show inhibition of microsomal NO metabolism by the heme poisons cyanide and CO (FIGS. 13A and 13B) and diphenyleneiodonium (DPI) (FIG. 13C).

FIGS. 14A and 14B show inhibition of microsomal NO metabolism by the NADPH-cytochrome P450 oxidoreductase (CYPOR) substrate-inhibitor cytochrome c.

FIGS. 15A and 15B show the effect of anti-CYPOR IgG on microsomal NO metabolism and cytochrome c reduction.

FIGS. 16A and 16B show the sensitivity of microsomal NO metabolism to Zn(II)-protoporphyrin.

### DETAILED DESCRIPTION

Nitric oxide dioxygenase (NOD) (EC 1.14.12.17) converts nitric oxide (NO) to nitrate and protects aerobic microbes from toxic NO. Inhibitors of NOD may be useful as antibiotics towards infectious microbes that utilize NOD as a protective stratagem against the immune system. Antifungal azoles have the capacity to inhibit NOD in vitro, to ligate the catalytic heme iron in NOD, and to inhibit NOD function within cells.

Azoles bound both the ferric and ferrous heme of NOD, as evidenced by UV-visible spectra, and showed non-competitive inhibition of NOD activity with respect to O₂ and NO. Azole binding impaired heme and flavin reduction by NADH. Miconazole inhibited NOD activity in S. cerevisiae and synergized with NO in inhibiting growth. Without being bound by a particular theory, the azoles may trap the ferric heme intermediate in the NOD reaction cycle. This provides an additional mechanism for antifungal action, as well as broader antimicrobial applications, for azoles.

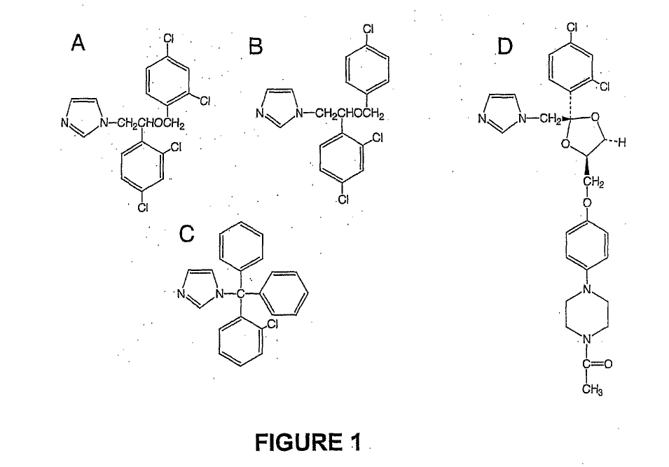

Each of miconazole, econazole, clotrimazole, and ketoconazole, shown in FIG. 1, inhibited microbial NOD activity.

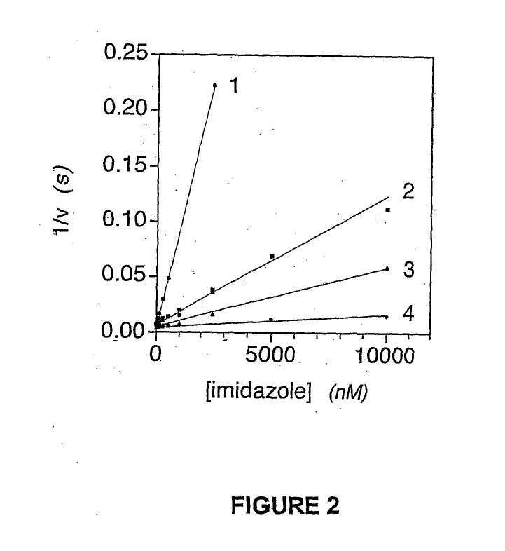

As shown in FIG. 2, imidazoles inhibited the activity of flavohemoglobin NOD isolated from E. coli. NOD activity was assayed at the indicated concentrations of the azoles miconazole (line 1), econazole (line 2), clotrimazole (line 3), and ketoconazole (line 4) with 200 μM O₂, 100 μM NADH, and 1 μM NO at 37° C. Miconazole was the most effective of the azoles tested in inhibiting NOD.

Inhibition of NOD by azoles was compared among E. coli, Alcaligenes eutrophus and Saccharomyces cerevisiae NODs, also flavohemoglobins, as shown in Table I.

Apparent Ki (nM)

| | E. coli | A. eutrophus | | S. cerevisiae |
|---|---|---|---|---|
| Imidazole | flavohemoglobin | flavohemoglobin | | flavohemoglobin |
| miconazole^{a} | 80 | 5^{c} | (˜70%) | 12,000 |
| | | 650 | (˜30%) | |
| econazole^{a} | 550 | 100^{c} | (˜70%) | 30,000 |
| | | 2,000 | (˜30%) | |
| clotrimazole^{a} | 1,300 | 200^{c} | (˜93%) | 50,000 |
| | | 5,000 | (˜7%) | |
| ketoconazole^{b} | 5,000 | 700^{c} | (˜75%) | >100,000 |
| | | 25,000 | (˜25%) | |

The solvents ^{a}DMSO and ^{b}methanol did not affect the activity at the final concentration of 0.1% (v/v). ^{c}Kᵢ values were obtained from biphasic profiles of 1/v vs. [imidazole], with the fraction inhibited given as a percentage of the total activity. Values are expressed in units of nM.

Apparent Kᵢ values in E. coli were 80 nM for miconazole, 550 nM for econazole, 1300 nM for clotrimazole, and 5000 nM for ketoconazole at 200 μM O₂, 1 μM NO, and 37° C. The specific activities of the E. coli, A. eutrophus and S. cerevisiae NOD were 185, 90 and 105 NO heme⁻¹ s⁻¹, respectively.

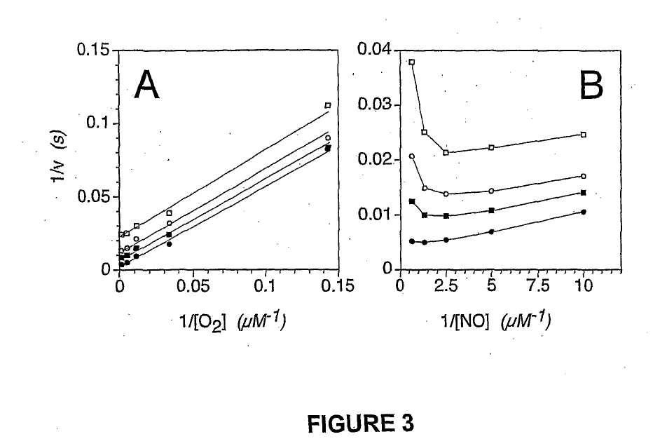

As shown in FIGS. 3A and 3B, NOD inhibition by miconazole was non-competitive with respect to O₂ and NO. Microbial NOD activity was assayed with varying concentrations of O₂ at 0.75 μM NO (A), and at varying concentrations of NO with 200 μM O₂ (B), in the presence of 0 μM (●), 0.1 μM (▪), 0.25 μM (∘), and 0.5 μM (□) miconazole at 37° C.

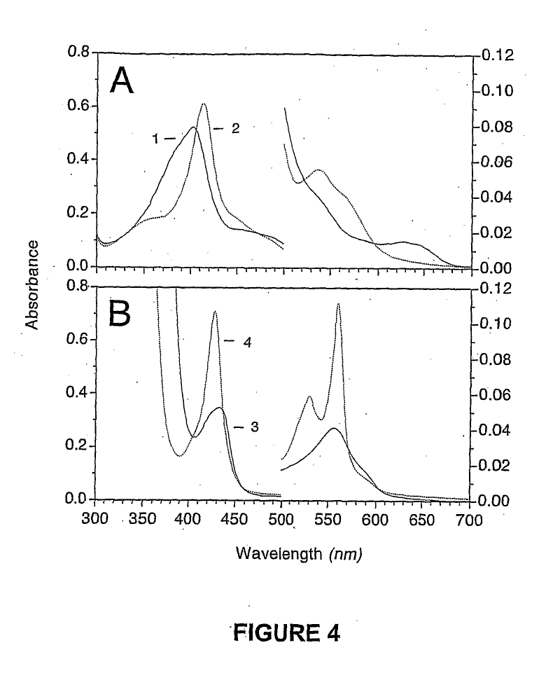

The spectra of the flavohemoglobin-miconazole complexes were analyzed. FIGS. 4A and 4B show oxidized (A) and reduced (B) flavohemoglobins and the corresponding miconazole flavohemoglobin complexes. Flavohemoglobin(FAD-Fe³⁺) (line 1), flavohemoglobin(FAD-Fe³⁺)-miconazole (line 2), flavohemoglobin(FADH2-Fe²⁺) (line 3), and flavohemoglobin(FADH2-Fe²⁺)-miconazole (line 4) spectra were recorded at room temperature in 100 mM sodium phosphate buffer, pH 7.0, containing 0.3 mM EDTA with 8.6 μM E. coli flavohemoglobin containing 5.9 μM heme and 8.6 μM FAD. Miconazole was added at a final concentration of 13 μM.

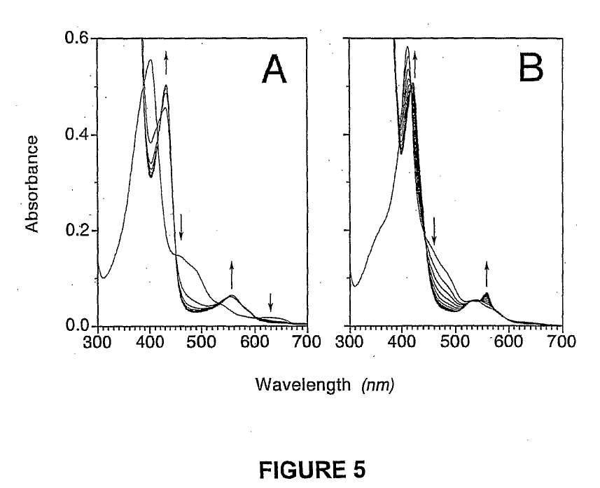

As shown in FIGS. 5A and 5B, miconazole inhibited reduction of the flavohemoglobin (Fe³⁺)-miconazole complex by NADH. Spectra of 8.6 μM E. coli flavohemoglobin containing 5.9 μM heme and 8.6 μM FAD were recorded at intervals in anaerobic buffer at 22° C. containing 1 mM NADH either in the absence (A) or presence (B) of miconazole. Miconazole was added at a final concentration of 13 μM prior to the addition of NADH. Arrows indicate increases or decreases in absorption maximal upon reduction with NADH.

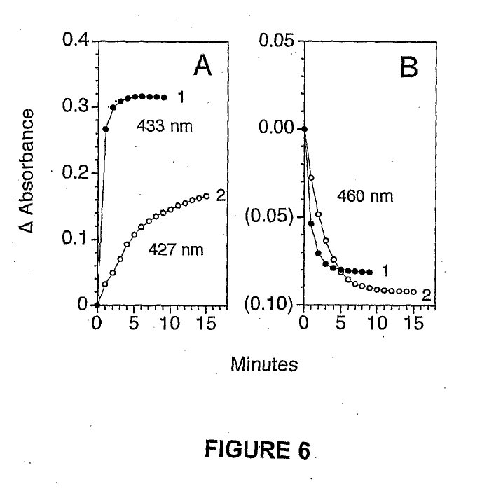

Miconazole inhibited heme and flavin reduction. FIG. 6A shows the formation of flavohemoglobin(FADH-Fe²⁺) as measured at 433 nm (heme Sorest) (line 1) and the flavohemoglobin(FADH-Fe²⁺)-miconazole complex as measured at 427 nm (line 2). FIG. 6B shows the reduction of bound FAD in the absence (line 1) or presence of miconazole (line 2), as measured at 460 nm.

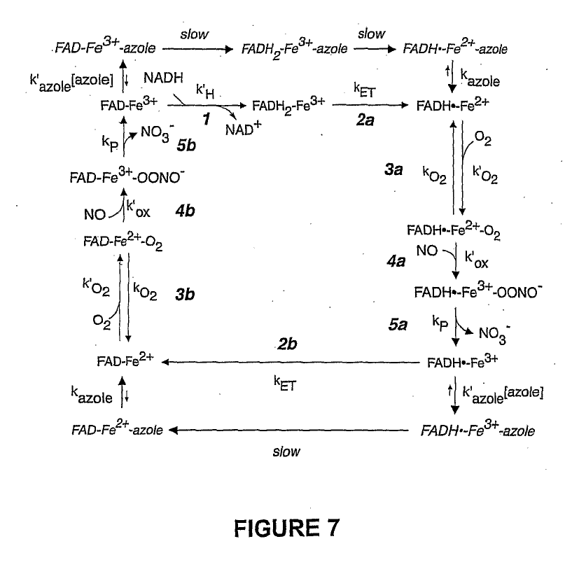

Without being bound by a particular theory, FIG. 7 shows a mechanism for imidazole inhibition of NOD. Imidazoles form legends with flavohemoglobin(FAD-Fe ³⁺) and flavohemoglobin(FADH-Fe³⁺) and inhibit hydride (reaction 1, k'H) and electron transfer (reactions 2a and 2b, k_{ET}). O₂ readily competes with azole for the reduced flavohemoglobin to form the active FADH-Fe²⁺O₂ and FAD-Fe²⁺-O₂ complexes.

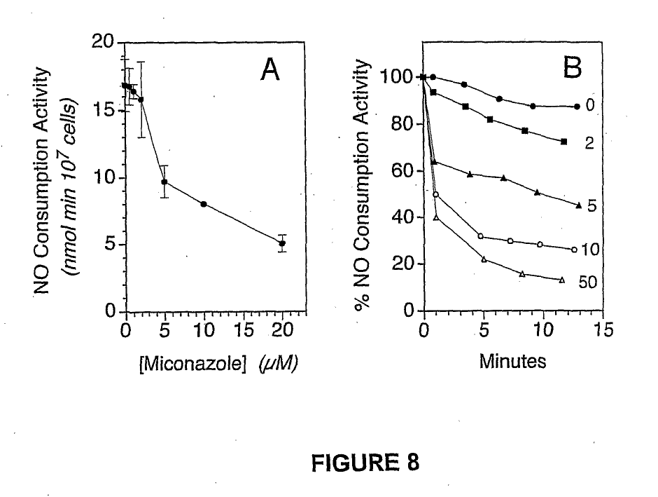

With respect to FIGS. 8A and 8B, miconazole inhibition of NOD activity in S. cerevisiae is shown. FIG. 8A shows NO consumption (NOD) activity of S. cerevisiae assayed with varying concentrations (0 μM to about 20 μM) of miconazole. Error bars represent the standard deviation of the average of three independent trials. FIG. 8B shows time-dependence of inhibition with miconazole at 0 μM (no miconazole), 2 μM, 5 μM, 10 μM, and 50 μM miconazole as indicated.

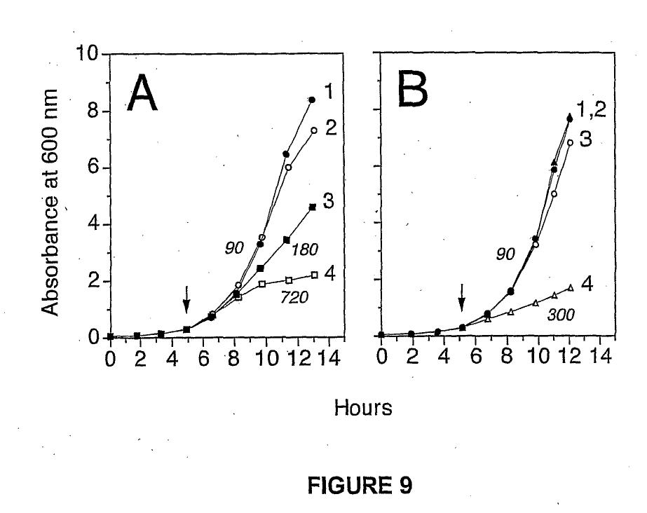

Miconazole and NO synergistically inhibited growth of S. cerevisiae, as shown in FIG. 9. In FIG. 9A, cultures of S. cerevisiae parental strain BY4742 were grown under an atmosphere containing 21% O₂ balanced with N₂. At the time indicated by the arrow, cultures were exposed to an atmosphere containing 960 ppm NO (<2 μM NO in solution) in a 21% O₂/N₂ balance (lines 2 and 4) or were maintained under an atmosphere of 21% O₂ balanced with N₂ (lines 1 and 3). Simultaneously, miconazole (5 μM) (lines 3 and 4) or DMSO solvent (0.01% v/v) only (lines 1 and 2) was added.

In FIG. 9B, parental strain BY4742 (lines 1 and 3) and flavohemoglobin deficient mutant ΔYHB1 (lines 2 and 4) were grown under an atmosphere containing 21% O₂ balanced with N2. At the time indicated by the arrow, cultures were either maintained fewer than 21% O₂ balanced with N2 (lines 1 and 3) or were exposed to 960 ppm NO in the 21% O₂/N₂-balanced atmosphere (lines 2 and 4). Cultures were grown and exposed to gases. Approximate generation times (min) are given in italics.

Without being bound by a specific theory, it is likely that azole binding to the ferric heme intermediate in NOD catalysis inhibited microbial NOD, rather than azole competing with O₂ for binding the ferrous heme. A single chlorine atom in miconazole (FIG. 1A) increased inhibition about 7-fold over that observed with econazole (FIG. 1B). This suggested specific interactions of phenyl group constituents within the conserved hydrophobic distal heme pocket, and a mechanism involving imidazole binding and trapping of the ferric heme intermediate in the NOD reaction cycle (FIG. 7). NOD is thus a likely target of the broad-spectrum antifungal and antibacterial imidazoles. Organisms lacking an alternative NO reductase pathway and preferentially utilizing a NOD pathway for survival are targets for NOD inhibition.

These imidazoles also inhibited the mammalian cell NOD, as will be subsequently described. Thus, heme-binding azoles may be engineered to specifically target NO metabolism and modulate NO functions in a variety of organisms substituting for NO modulation therapies employing NO delivery agents. Mechanistic inhibitors of mammalian NOD have application as anti-tumor agents and vasorelaxants (Hallstrom et al. Free Radic. Biol. Med. 37(2) (2004)), which is expressly incorporated by reference herein in its entirety). NO catabolic pathways may also provide immune resistance to carcinomas, and thus serve as novel targets for cancer intervention. In addition, O₂ dependent NO decomposition catalysts may provide a dynamic feedback mechanism for modulating homeostatic NO levels in tissues (and O₂ delivery by capillaries) in response to the prevailing steady-state O₂ concentrations in tissues. Inhibitors of NOD, by inhibiting NO decomposition, may increase NO signaling and O₂ delivery. Inhibition of NOD activity may be partly responsible for the NO-dependent relaxation of arterioles noted for agents such as allicin or carbon monoxide (CO).

In addition to azoles, other heme ligands inhibit the flavohemoglobin-catalyzed NOD reaction and the mammalian NOD activity. Cyanide inhibits microbial (flavohemoglobin) NOD and the mammalian NOD at low micromolar concentrations, suggesting a common mechanism involving the high affinity binding of cyanide to the ferric heme. Cyanide also serves as a useful agent for determining heme enzyme or flavohemoglobin involvement in cellular NO metabolic activities.

CO shows high affinity for the ferrous hemes of flavohemoglobins with dissociation equilibrium constants of less than 0.7 μM, and shows strong competitive inhibition of NOD activities with respect to O2 (Kᵢ=about 1 μM). CO similarly inhibits the NOD activity in mammalian cells (Kᵢ=about 3 μM) suggesting a flavohemoglobin-like mechanism for that activity.

Allicin (diallyl thiosulfinate) is a medically active compound formed by reaction of the enzyme allinase with the amino acid alliin (S-allylcysteine suffixed) when garlic is crushed. Allicin has diverse antimicrobial effects, such as antibacterial activity against a wide range of Gram negative and Gram-positive bacteria, antifungal activity, ant parasitic activity, and antiviral activity. The main antimicrobial effect of allicin has been reported to result from its chemical reaction with thiol groups of various enzymes, and it has been reported to transiently deplete cellular glutathione levels. Allicin also reacts with and modifies heme in cytochrome P450 enzymes such as the 2C9 and 2C19 isoforms. Allicin potently inhibits NODs within mammalian cells and bacteria. Allicin also inactivates the isolated E. coli NOD. Phytoanticipins such as amygdalin found in almonds, cherry, and peach kernels, and phytoalexins may also be used.

Human intestinal Caco-2 cells metabolized and detoxified NO via a dioxygen- and NADPH-dependent cyanide- and CO-sensitive pathway that yielded nitrate. Enzymes catalyzing NO dioxygenation fractionated with membranes and were enriched in microsomes. Microsomal NO metabolism showed apparent KM values for NO, O₂, and NADPH of 0.3 μM, 9 μM, and 2 μM, respectively, values similar to those determined for intact or digitonin-permeabilized cells. Similar to cellular NO metabolism, microsomal NO metabolism was superoxide-independent and sensitive to heme-enzyme inhibitors including CO, cyanide, imidazoles, quercetin, and allicin-enriched garlic extract.

Selective inhibitors of several cytochrome P450s and heme oxygenase failed to inhibit the activity, indicating limited roles for a subset of microsomal heme enzymes in NO metabolism. Diphenyleneiodonium (DPI) and cytochrome c(III) inhibited NO metabolism, suggesting a role for the NADPH-cytochrome P450 oxidoreductase (CYPOR). Involvement of CYPOR was demonstrated by the specific inhibition of the NO metabolic activity by inhibitory anti-CYPOR IgG. The results suggested roles for a microsomal CYPOR-coupled and heme-dependent NO dioxygenase in NO metabolism, detoxification, and signal attenuation in mammalian cells and tissues.

Human colorectal epithelial adenocarcinoma Caco-2 (HTB-37) and the human epithelial-like lung adenocarcinoma A549 (CCL185) (American Type Culture Collection (Rockville, Md.)) were used. Reagents were obtained from Sigma-Aldrich Fine Chemicals (St. Louis, Mo.) unless otherwise indicated. Anti-CYPOR goat IgG (4.4 mg per ml) was kindly provided by Dr. Bettie Sue Masters (Univ. Texas, San Antonio). Bovine erythrocyte copper, zinc-superoxide dismutase (Cu,ZnSOD) (5000 U per mg), Aspergillus nitrate reductase (10 U per mg), bovine liver catalase (260,000 U per ml) and digitonin were from Roche Molecular Biochemicals (Indianapolis, Ind.). Protoporphyrin IX, Zn(II)-protoporphyrin IX and Sn(IV)-protoporphyrin IX were from Frontier Scientific, Inc. (Logan, Utah). Cytochrome c(II) was prepared by reducing 40 mg of cytochrome c(III) in 1 ml of buffer containing 50 mM Tris-Cl, pH 8.0 and 1 mM EDTA with sodium dithionite and dialyzing extensively against the same buffer. Cytochrome c(III) and cytochrome c(II) concentrations were determined by absorbance at 550 nm applying respective extinction coefficients of 8.9 and 29.9 mM-1 cm-1. Cylinders of ultra-pure N2 (99.998%), O₂ (99.993%) and CO (99.5%) gases were from Praxair (Bethlehem, Pa.). NO gas (98.5%) was from Sigma-Aldrich Fine Chemicals. Saturated NO (about 2 mM), CO (1 mM) and O₂ (1.14 mM) stocks were prepared as previously described in Gardner, P. R. et al., Free Rad. Biol. Med. 31:191-204; 2001; and Gardner, P. R. et al., 2004, Nitric Oxide Protocols, vol. 279. A. Hassid, Ed., Humana Press, Totowa, N.J. 133-150, each of which is expressly incorporated by reference herein. Garlic extract was prepared by homogenizing 160 grams of fresh garlic cloves (Allium sativum) with 100 ml of water in a blender and incubating the homogenate at 37° C. for one hour to allow the enzymic formation of allicin. Homogenates were filtered through a cheese cloth to remove large tissue debris, centrifuged at 30,000 g for 30 minutes to clarify, and were extracted with an equal volume of chloroform. Separation was facilitated by centrifugation at 2000 g for 10 minutes, and the chloroform extract was collected. Chloroform was evaporated by sparging with air yielding about 0.5 ml of an allicin-enriched oil that was stored neat and as a 1% emulsion in water at −80° C.

Cells were grown, harvested and counted as previously described (Gardner, P. R. et al., Free Rad. Biol. Med. 31:191-204; 2001). Cells were either resuspended for immediate assay of NO metabolism or were stored frozen at −80° C. for fractionation studies. Cells were fractionated as described (Gardner, P. R. et al., 2004, Nitric Oxide Protocols, vol. 279. A. Hassid, Ed., Humana Press, Totowa, N.J. 133-150). Protein was measured using Peterson's modification to the Lowry method with bovine serum albumin as the standard.

Rates of NO consumption by Caco-2 cells were measured in DPBS containing 5 mM glucose and 100 μg/ml cycloheximide (Gardner, P. R. et al., Free Rad. Biol. Med. 31:191-204; 2001; and Gardner, P. R. et al., 2004, Nitric Oxide Protocols, vol. 279. A. Hassid, Ed., Humana Press, Totowa, N.J. 133-150). Initial rates of NO consumption were measured at 1 μM NO unless otherwise stated, and all rates were corrected for background rates of NO decomposition. A milliunit of activity is defined as the amount metabolizing 1 nanomol NO per min. For measurements of CO inhibition and the reversibility by white light, cells (2.5×105) were either kept in the dark or illuminated with a Eastman Kodak Model 4400 slide projector (Eastman Kodak, Rochester, N.Y.) equipped with a 300 W tungsten lamp and no external lens and set at a distance of about 16 cm from the internal lens. Sensitivity to CO was measured with 12.5 μM O₂. Rotenone (0.5 μM) was included in CO inhibition assays to block respiration and O2 depletion. For measurements of the NADPH dependence of cellular NO consumption, Caco-2 and A549 cells were permeabilized with 0.0025% (w/v) digitonin in 100 mM Na-Hepes, pH 7.8 containing 0.25 M sucrose and 30 μM Cu,ZnSOD. Cell permeabilization was monitored by the loss of NO metabolic activity.

NO metabolism by cell fractions was assayed in 100 mM Na-Hepes, pH 7.8, 0.25 M sucrose, 1 mM EDTA and 1 mM EGTA (Sucrose Buffer) containing 15 μM Cu,ZnSOD and 100 μM NADPH. Cell fractions were added with a 50 μl Hamilton syringe to give a total of 100-750 μg protein. For determination of O₂ dependence of microsomal NO metabolism, the 2 ml reaction was sparged with N₂ for 10 minutes to remove O₂, and O₂ was depleted from microsomal membranes by stirring membranes under a stream of N₂ in a rubber septum-sealed tube on ice. Alternatively, residual O₂ was removed by incubating the reaction mix with 16 units glucose oxidase, 1 mM glucose and 260 units catalase for 5 minutes prior to adding NO and microsomes. O₂ was added from O₂ saturated buffer to achieve various O₂ concentrations.

Nitrite and nitrate were assayed using the Griess reaction essentially as described for whole cells (Gardner, P. R. et al., Free Rad. Biol. Med. 31:191-204; 2001; and Green, L. C. et al., Anal. Biochem. 126:131-138; 1982, expressly incorporated by reference herein in its entirety). Microsomes possessing about 7.5 mU of NO metabolic activity were added to the 2 ml reaction chamber in Sucrose Buffer containing 15 μM Cu,ZnSOD, 100 μM NADPH followed by addition of 20 μl of NO from fresh NO-saturated water stocks. NO was injected over the course of 6 min such that the NO concentration never exceeded about 0.7 μM. Reaction products were collected, centrifuged to remove membranes, and the supernatant was assayed for nitrite and nitrate.

CYPOR (cytochrome c reductase activity) was measured by following the initial rate of cytochrome c reduction by microsomes or purified enzyme at 37° C. in 1 ml of Sucrose Buffer containing 100 μM NADPH, 15 μM Cu,ZnSOD and 20 μM cytochrome c(III) unless otherwise indicated. A unit of CYPOR activity was defined as the amount reducing one μmol of cytochrome c per minute.

To determine CYPOR, 1,000 g membrane (342 μg protein), 10,000 g membrane (140 μg protein) and 20,000 g membrane (342 μg protein) fractions were incubated on ice for 2 hrs with either bovine serum albumin (BSA) (132 μg), anti-CYPOR IgG (132 μg), anti-CYPOR IgG (132 μg) plus CYPOR (2.6 μg) or isotype-matched IgG (132 μg) in a total volume of 150 μl. The incubations contained 120 μl of Sucrose Buffer and 30 μl of PBS (8.1 mM Na2HPO4, 1.1 mM KH2PO4, 138 mM NaCl and 2.7 mM KCl, pH 7.4) introduced with IgG or BSA. Anti-CYPOR was tested at ratios to CYPOR activity capable of producing about 60% to about 80% inhibition of purified CYPOR.

The Tukey-Kramer HSD statistical analysis method in the program JMP (SAS Institutes, Inc., Cary, N.C.) was used for the analysis of significance (p<0.05).

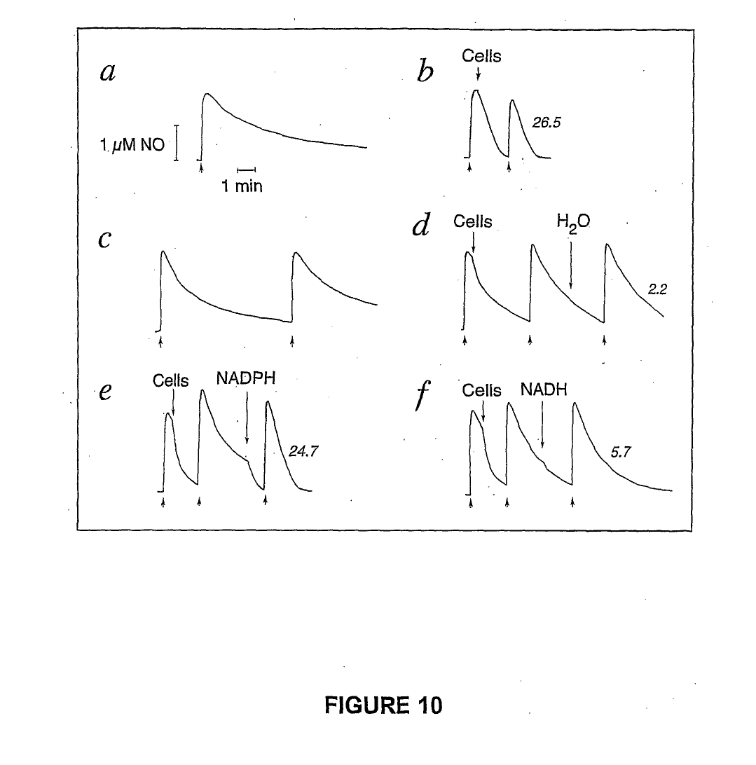

NO metabolism by permeabilized mammalian cells was determined. As shown in FIG. 10, human Caco-2 cells metabolized NO robustly (compare trace b with background trace a). Cells were gently permeabilized with digitonin to determine substrate and cofactor requirements of the NO metabolic activity. Background NO decomposition (trace a) and NO consumption by intact Caco-2 cells (1.0×106) (trace b) was measured in DPBS containing glucose and 200 μM O₂. Background NO decomposition (trace c) and NO consumption by digitonin-permeabilized cells (1.0×106) (traces d-f) was measured in 100 mM sodium Hepes buffer, pH 7.8, containing 0.25 M sucrose, 30 μM Cu,ZnSOD, 200 μM O₂ and 0.0025% (w/v) digitonin. Solvent water (2 μl) (trace d), NADPH (100 μM) (trace e) or NADH (100 μM) (trace f) were added during the course of cell permeabilization. Arrows denote addition of 2 μM NO. Initial rates were determined at 1 μM NO and are given in italics as nmoles NO per min per 107 cells with correction for background rates. Data are representative of two or more trials.

Progressive and greater than 90% loss of activity followed three successive additions of NO (trace d). The activity was fully recovered by addition of 100 μM NADPH (trace e) and showed an apparent KM(NADPH) value of 0.8 μM with 1 μM NO at 200 μM O₂ (data not shown). In contrast, <20% of the activity was recovered with 100 μM NADH (trace D. A similar preference for NADPH was observed using digitonin-permeabilized human lung A549 cells (data not shown).

The results demonstrated the dependence of the NO metabolic activity upon NADPH, a minimal effectiveness of NADH, and no additional requirement for diffusible cofactors. The results did not, however, exclude the involvement of lipids or other membrane bound cofactors.

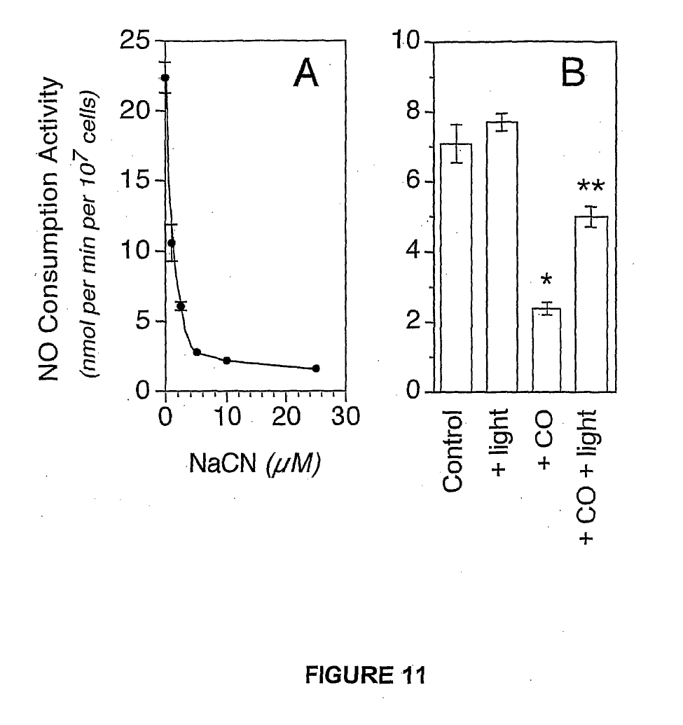

With respect to FIGS. 11A and 11B, the sensitivity of cellular NO metabolism to heme enzyme inhibitors and free radical scavengers was determined. NO consumption by Caco-2 cells was assayed in the presence of varying concentrations of NaCN with 200 μM O₂ (FIG. 11A) or in the presence or absence of 5 μM CO with 12.5 μM O₂ (FIG. 11B). Reactions were kept in the dark (Control) or illuminated (+Light) as previously described. * indicates p<0.05 relative to Control. ** indicates p<0.05 relative to +CO and +Light. Error bars represent the SD of three independent trials.

As shown in FIG. 11A, cyanide inhibited dioxygen-dependent NO metabolism in various mammalian cells. Half-maximal inhibition of the activity in Caco-2 cells occurred with <2 μM NaCN. As shown in FIG. 11B, the activity was also competitively inhibited by the ferrous heme ligand CO at a CO:O₂ ratio of about 1:5 (Kᵢ(CO)=3 μM), and this inhibition was rapidly reversed by exposure of cells to white light. The sensitivity of NO metabolism to cyanide and the light-reversible CO inhibition support mechanisms of inhibition involving binding of CO and cyanide to a catalytic heme similar to the microbial NOD (flavohemoglobin). The light reversible CO inhibition was also reminiscent of that described for xenobiotic-metabolizing cytochrome P450s.

In addition to cyanide and CO, heme-binding imidazoles, a panel of substrate-inhibitors of cytochrome P450s, inhibitors of the NO-binding heme oxygenases, and free radical scavengers were surveyed for effects on NO metabolism by Caco-2 and A549 cells. All agents were used at concentrations showing minimal cytotoxicity as defined by <5% decrease in trypan blue exclusion following 15 min exposure (data not shown). Caco-2 and A549 cells were grown, harvested and assayed for NO consumption activity following a 15 min incubation in 2 ml DPBS containing 5 mM glucose and 100 μg/ml cycloheximide with the indicated agent as previously described. Data represent the mean ±SD of three independent exposures. Bold numbers indicate p<0.05 relative to the control. 100% activity is equivalent to 25.4±1.4 and 12.5±1.5 nmol NO per minute per 107 cells (n=6) for Caco-2 and A549 cells, respectively. The results are show in Table II.

TABLE II
Effects of Heme Enzyme Inhibitors and Radical
Scavengers on Cellular NO Metabolism

% Activity

| Agent | Caco-2 | A549 |
|---|---|---|
| NaCN, 100 μM ^{a} | 5.0 ± 0.8 | 9.3 ± 4.6 |
| ketoconazole, 100 μM ^{b} | 22.3 ± 4.1 | 23.1 ± 3.9 |
| miconazole, 20 μM ^{c} | 37.1 ± 2.4 | 38.2 ± 5.9 |
| econazole, 20 μM ^{c} | 43.0 ± 3.6 | 36.2 ± 4.9 |
| clotrimazole, 100 μM ^{c} | 29.2 ± 0.7 | 31.5 ± 2.3 |
| metronidazole, 100 μM ^{c} | 80.2 ± 8.0 | 79.7 ± 2.9 |
| troleandomycin, 100 μM ^{c} | 98.0 ± 3.5 | 92.9 ± 3.2 |
| erythromycin, 100 μM ^{c} | 97.1 ± 7.8 | 94.6 ± 0.4 |
| furafylline, 10 μM ^{c} | 93.8 ± 6.9 | 98.2 ± 3.1 |
| sulfaphenazole, 100 μM ^{c} | 97.1 ± 3.0 | 95.6 ± 5.5 |
| quercetin, 100 μM ^{c} | 69.2 ± 3.9 | 75.6 ± 1.8 |
| β-naphthoflavone, 100 μM ^{c} | 72.8 ± 3.7 | 85.1 ± 0.6 |
| diallyl sulfide, 100 μM ^{d} | 98.1 ± 1.7 | 98.1 ± 3.3 |
| garlic extract, 10 μg/ml ^{a} | 16.0 ± 2.3 | 14.4 ± 3.2 |
| quinidine, 100 μM ^{c} | 93.7 ± 7.0 | 85.5 ± 9.0 |
| Zn2+-protoporphyrin, 100 μM ^{c} | 64.0 ± 2.4 | 56.5 ± 8.0 |
| Sn4+-protoporphyrin, 100 μM ^{c} | 101.8 ± 1.8 | 100.0 ± 1.9 |
| L-NAME, 1 mM ^{a} | 98.2 ± 1.2 | 101.0 ± 2.9 |
| α-tocopherol, 100 μM ^{d} | 98.8 ± 3.6 | 94.1 ± 6.9 |
| BHT, 100 μM ^{d} | 92.4 ± 5.6 | 92.2 ± 5.9 |

^{a} Agents were dissolved in water.
^{b, c, d} The solvents methanol, DMSO and ethanol were introduced at 0.1% (v/v), respectively. None of the solvents significantly affected the NO consumption activity.

Ketoconazole, miconazole, econazole, clotrimazole and metronidazole each inhibited the NO metabolic activity to similar extents within Caco-2 and A549 cells. In contrast, substrate-inhibitors of microsomal cytochrome P450 (CYP) isozymes CYP1A1 (βnaphthoflavone and quinidine), CYP1A2 (furafylline), CYP3A4 (erythromycin and troleandomycin), CYP2E1 (diallyl sulfide), CYP2C9 (sulfaphenazole), CYP2D6 (quinidine) and NO synthase (N ω-nitro-L-arginine methyl ester) (L-NAME) did not consistently affect the NO consumption activity within these two cell types. The activity was moderately sensitive to inhibition by the heme-binding flavonoid and cytochrome P450 enzyme inhibitor quercetin (a CYP1A1, CYP2C9 and CYP2C19 inhibitor), but was strongly inhibited by a garlic extract enriched in allicin (a CYP2C9 and CYP2C19 inhibitor and a NO-dependent vasodilator). Zn(II)-protoporphyrin added at 100 μM inhibited the NO metabolic activity by about 40% in Caco-2 and A549 cells. However, the more potent heme oxygenase inhibitor, Sn(IV)-protoporphyrin at 100 μM (Kᵢ=<100 nM), showed no effect on the activity. Similar effects of Zn(II)-protoporphyrin and Sn(IV)-protoporphyrin were observed in the dark (data not shown). Thus, the ability or inability of porphyrins to inhibit was not dependent upon light.

These results demonstrated a role for a heme enzyme in Caco-2 and A549 NO consumption, but suggested limited roles for heme oxygenase, NO synthase, and cytochrome P450 isozymes CYP1A1, CYP1A2, CYP2C9, CYP2D6, CYP2E1, and CYP3A4. Caco-2 cells reportedly express heme oxygenase, CYP1A1, CYP2D6, CYP3A4 and CYP3A5 isozymes, and A549 cells express CYP1A1, CYP1B1, CYP2B6, CYP2C, CYP2D6, CYP2E1, CYP3A5, but not CYP3A4. The activity was not inhibited by α-tocopherol or butylated hydroxytoluene (BHT), indicating a limited role for lipid peroxidation products including peroxyl and alkyl radicals in cellular NO metabolism. The results were consistent with the negligible role of H₂O₂ in NO metabolism.

The metabolism of NO by microsomal membranes is shown in Table III. Caco-2 cells (˜7×108) were homogenized and fractionated by differential centrifugation and fractions were assayed for protein and NOD activity as previously described. In parentheses, m=membranes and s=soluble supernatant. Data represent the average (±SD) of three independent fractionations.

TABLE III
Subcellular Fractionation of Caco-2 NOD Activity

| | Total | Total | | Specific |
|---|---|---|---|---|
| | Protein | Activity | | Activity |
| Fraction | mg | mU | (%) | mU/mg |
| Homogenate | 360 ± 20 | 1836 ± 102 | 100 | 5.1 ± 0.1 |
| 1,000 g (m) | 212 ± 19 | 1272 ± 114 | 69 | 6.0 ± 0.2 |
| 10,000 g (m) | 24 ± 3 | 400 ± 23 | 9 | 7.1 ± 0.2 |
| 20,000 g (m) | 8.6 ± 1.2 | 94 ± 13 | 5 | 10.9 ± 0.4 |
| 20,000 g (s) | 103 ± 6 | 6.1 ± 0.4 | 0.3 | 0.06 ± 0.04 |

Homogenization of Caco-2 cells and fractionation of components by differential centrifugation revealed a distinct distribution of the NADPH-dependent NO metabolic activity with membranous organelles. The highest specific activity was measured in the low density (20,000 g) membrane fraction corresponding to microsomal membranes derived from the endoplasmic reticulum. A significant fraction of the activity was also detected in the denser membrane fractions, however, the specific activity of these denser membrane fractions containing primarily trypan blue-permeable cell ghosts and nuclei (1,000 g), and mitochondria (10,000 g), respectively, were invariably lower and most likely contain membranes derived from the endoplasmic reticulum. The NO metabolic activity measured for intact Caco-2 cells, 4.8±0.3 mU/mg cell protein or about 1,728 m/U for 360 mg of total cell protein, was fully recovered in the homogenate and the membrane fraction. Furthermore, the activity in each fraction was inhibited ≧90% by 100 μM NaCN (data not shown).

As with permeabilized cells, 100 μM NADH supported about 20% of the microsomal activity observed with NADPH (data not shown). Moreover, Cu,ZnSOD (15 μM ) did not significantly affect the rate of NO metabolism by the microsomes (data not shown). Under these conditions, microsomes catalyzed the decomposition of about 40 nmol NO (20 μl of about 2 mM NO) to 36.6±6.7 nmol nitrate plus nitrite with 97±3% of the product of reactions being nitrate (n=3, ±SD). Thus, microsomal NO metabolism was NADPH-dependent, superoxide-independent, and produced predominantly nitrate. The NO metabolic activities of various microsomal membrane preparations were up to 16-fold higher than the activity previously measured in sonic extracts of Caco-2 cells (0.8 mU/mg protein). Loss of activity during microsome preparation, freezing and thawing may have accounted for the lower specific activities of some microsome preparations.

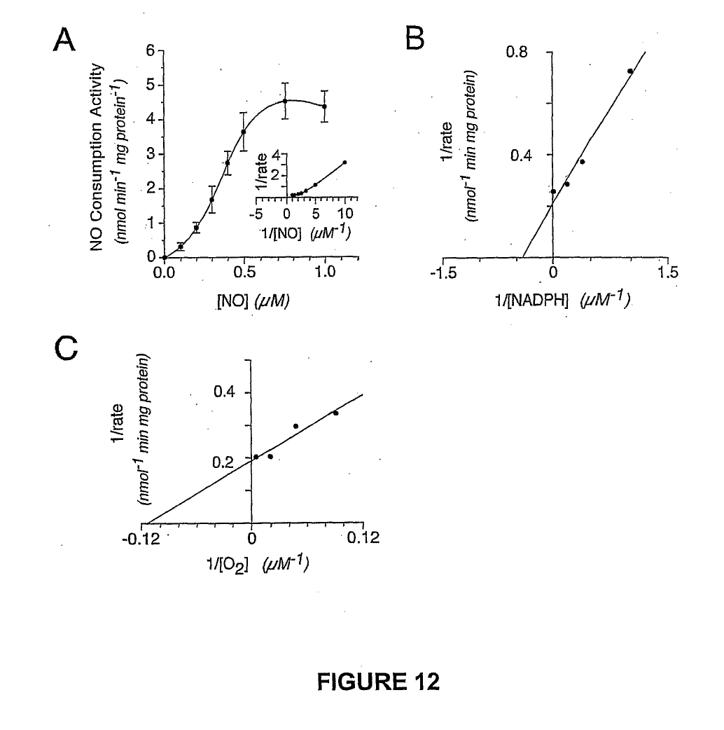

The effects of NO, O₂ and NADPH dependence on microsomal metabolism are shown in FIG. 12. FIG. 12A shows NO dependence of NO consumption measured with 200 μM O₂ and 100 NADPH. Error bars represent the average±SD of five trials. (Inset) Plot of 1/v vs. 1/[NO] showing deviation from Michaelis-Menten kinetics. FIG. 12B shows NADPH dependence measured for 1 μM NO and 200 μM O₂ (C). O₂ dependence was measured with 100 μM NADPH at 1 μM NO. Data in panels B-C represent averages of three independent trials. Linear fits were achieved using Cricket Graph III (Computer Associates, Inc.).

Microsomal NO metabolism showed complex kinetics with respect to the concentration of NO. The reaction showed cooperativity at <0.5 μM NO and saturation-inhibition by NO at >0.5 μM NO. Half-maximal activity was observed with 0.3 μM NO. Cells showed similar NO inhibition and non-linear Lineweaver-Burk plots. NO metabolism was NADPH and O₂ dependent. Removal of O₂ from the reaction with glucose oxidase and catalase completely eliminated the NO metabolic activity of microsomes. Apparent KM values for NADPH and O₂ of 2 μM and 9 μM, respectively, were estimated from the Lineweaver-Burk plots in FIGS. 12B and 12C.

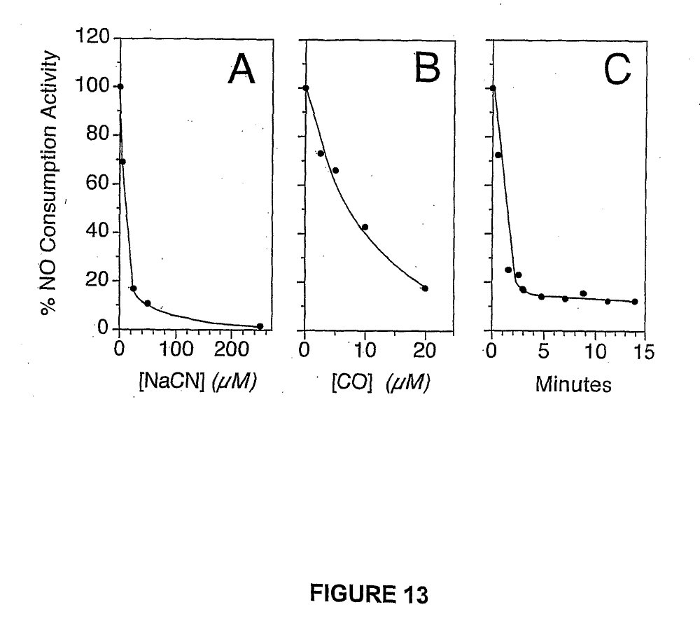

Similar to the activity in intact cells, the microsomal activity was potently inhibited by the heme enzyme poisons cyanide and CO, as shown in FIG. 13. In FIG. 13A, NaCN was tested for inhibition of NO consumption by microsomal membranes at 200 μM O₂ and 1 μM NO at varying concentrations. In FIG. 13B, the effects of concentrations of CO were measured at 20 μM O₂ and 1 μM NO. In FIG. 13C, NO metabolism was assayed at intervals in the presence of 50 μM DPI following repeated additions of NO. Percent activity was calculated relative to a DMSO (0.1% v/v) solvent control. 100% activity was equal to about 4 nmol NO per min per mg protein. Data represent the average of two or more two independent trials.

Greater than 80% inhibition was observed with 20 μM NaCN (FIG. 13A), and CO competitively inhibited the activity with respect to O₂. At 20 μM O₂, 10 μM CO inhibited the activity by >50% (FIG. 13B). In addition, other agents that inhibited NO metabolism by Caco-2 and A549 cells (Table II) also inhibited NO metabolism by Caco-2 microsomal membranes. Ketoconazole, miconazole, econazole and garlic extract inhibited the microsomal activity at relatively low concentrations and to extents similar to those observed in cells (Table IV). NO consumption by Caco-2 microsomes was assayed as previously described. Data represent the mean±SD of three measurements. Bold numbers indicate p<0.05 relative to the control. IC50 is the concentration that inhibited by 50%.

TABLE IV
Effects of Heme Enzyme Inhibitors and Radical
Scavengers on Microsomal NO Metabolism

| Inhibitor | % Activity | IC₅₀ | |
|---|---|---|---|
| ketoconazole, 100 μM ^{a} | 25.9 ± 2.6 | 10 | μM |
| miconazole, 100 μM ^{b} | 17.0 ± 0.0 | 20 | μM |
| econazole, 100 μM ^{b} | 15.3 ± 0.0 | 10 | μM |
| quercetin, 100 μM ^{b} | 84.1 ± 4.4 | n.d. | |
| garlic extract, 10 μg/ml ^{c} | 29.7 ± 2.4 | <10 | μg/ml |

| | | |
|---|---|---|
| L-NAME, 1 mM ^{c} | 96.7 ± 6.0 | n.d. |
| α-tocopherol, 100 μM ^{d} | 106.9 ± 3.4 | n.d. |
| BHT, 100 μM ^{d} | 94.8 ± 3.4 | n.d. |

^{a, b, d} The solvents methanol, DMSO and ethanol were introduced at 0.1% (v/v), respectively.
^{c} Agents were dissolved in water. Solvents alone did not significantly affect the NO consumption activity.
n.d. = not determined.

Quercetin showed more modest inhibition of the microsomal activity. The activity was not inhibited by α-tocopherol or BHT indicating a limited role for lipid peroxidation products in NO scavenging. The results demonstrated that the cellular NO metabolic activity, a nitrate-producing heme-dependent NOD, co-fractionated with microsomal membranes.

The microsomal NO metabolic activity was rapidly inactivated by DPI, an inhibitor of flavoenzymes including the endoplasmic reticulum and nuclear envelope-localized CYPOR. Seventy-five percent of the activity was lost within two minutes of exposure to 50 μM DPI (FIG. 13C). The effect of DPI was similar to that previously reported for intact Caco-2 cells.

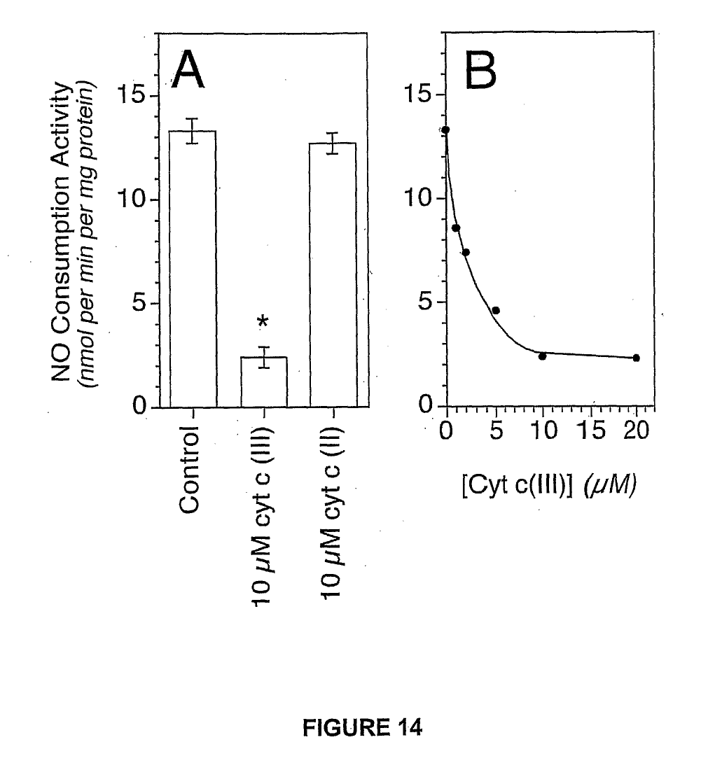

As shown in FIG. 14, cytochrome c(III), a substrate and inhibitor of CYPOR (apparent Ki=about 1 μM), also transiently inhibited microsomal NO metabolism, whereas reduced cytochrome c (10 μM) showed no effect on the activity. In FIG. 14A, oxidized and reduced cytochrome c (10 μM) was tested for inhibition of microsomal NO consumption. FIG. 14B shows the effect of cytochrome c(III) concentration on the activity. Initial NO consumption rates were assayed within 30 seconds of adding cytochrome c. Error bars in FIG. 14A represent the SD from the mean for three independent trials. Data in FIG. 14B represent the average of two trials. * indicates p<0.05 relative to the Control.

Fifty percent inhibition of microsomal NO metabolism was observed with about 2.5 μM cytochrome c(III) (FIG. 14B). Neither oxidized or reduced cytochrome c alone affected NO decomposition rates, suggesting limited reactivity of NO with cytochrome c under these conditions. Reduction of cytochrome c by membranes occurred rapidly under these conditions (data not shown) and can explain the transient inhibition. Further, microsomes showed a low rate of superoxide dismutase-sensitive cytochrome c reduction (<0.6 nmol min per mg protein) (data not shown) demonstrating a negligible role for superoxide radical in NO metabolic activity.

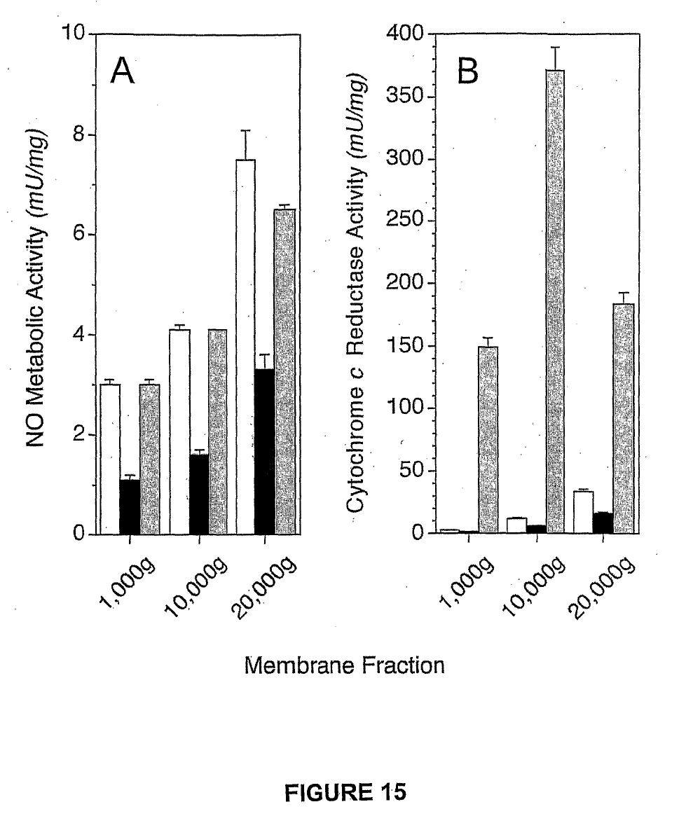

The role of CYPOR in NADPH-dependent NO metabolism and cytochrome c reduction by membrane fractions was tested directly with inhibitory anti-CYPOR IgG (FIG. 15). Caco-2 membrane fractions were incubated with BSA (open bars), anti-CYPOR IgG (solid bars), or anti-CYPOR plus recombinant human CYPOR (shaded bars). NO metabolism FIG. 15A and cytochrome c reduction FIG. 15B were assayed as previously described. Error bars represent the SD of the mean for three independent trials.

Anti-CYPOR IgG inhibited the NO metabolic activity and the cytochrome c reductase activity of the high-density membrane fractions (1,000 g and 10,000 g) and the low-density microsomal membranes (20,000 g) to similar extents (FIG. 15). Moreover, addition of recombinant human CYPOR to the antibody reaction relieved the inhibition of NO metabolic activity by anti-CYPOR IgG in all cases, while CYPOR alone did not support NO metabolism (FIG. 15A, compare open bars and shaded bars). Further, IgG from non-immune goats shows a minimal 13±4% inhibition of the microsomal activity relative to that observed for anti-CYPOR IgG (61±2%). Together, the results demonstrated an essential role for CYPOR in microsomal and cellular NO metabolism and suggested a mechanism involving CYPOR coupling of NADPH oxidation to reduction of cytochrome P450, heme oxygenase or another microsomal heme enzyme.

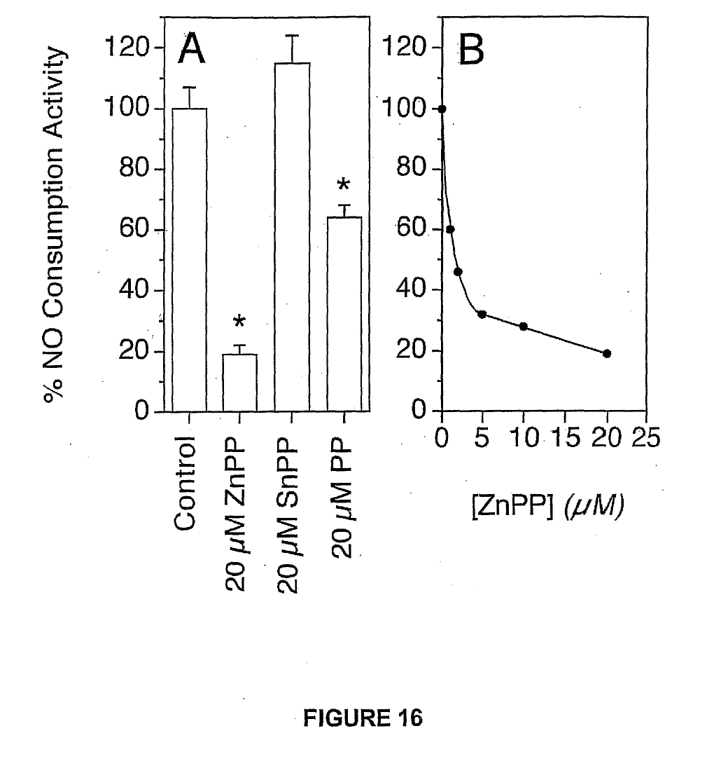

Zn(II)-protoporphyrin inhibited microsomal NO metabolism and CYPOR. In FIG. 16A, NO consumption was assayed with no addition (Control), 20 μM Zn(II)-protoporphyrin (ZnPP), 20 μM Sn(IV)-protoporphyrin (SnPP) or 20 μM protoporphyrin (PP). In FIG. 16B, NO consumption was assayed using varying concentrations of Zn(II)-protoporphyrin. 0.1% (v/v) DMSO was present as the solvent in all reactions. 100% activity was equal to 13.5 nmol NO per min per mg protein. Error bars in FIG. 16A represent the SD of the mean for three independent trials, Data in FIG. 16B represent the average of two trials. ** indicates p<0.05 relative to Control.

As in intact cells (Table II), Zn(II)-protoporphyrin (20 μM), but not Sn(IV)-protoporphyrin (20 μM), inhibited microsomal NO metabolism (FIG. 16A) demonstrating a limited role for heme oxygenase. The non-metallated protoporphyrin also inhibited NO metabolism to a significant (p<0.05), albeit lesser, extent (FIG. 16A). Again, porphyrins showed a similar pattern of inhibition in the dark (data not shown).

Inhibition of microsomal NO metabolism by Zn(II)-protoporphyrin was progressive, with 50% inhibition occurring with <2 μM Zn(II)-protoporphyrin (FIG. 16B). Zn(II)-protoporphyrin (20 μM) and protoporphyrin (20 μM) caused a similar progressive inhibition of cytochrome c reduction by microsomal membranes and purified CYPOR (data not shown), suggesting that these porphyrins interfered with CYPOR-mediated reduction of the microsomal heme enzyme. Sn(IV)-protoporphyrin showed strong interfering absorption at 550 nm and was not tested for effects on cytochrome c reduction.

In contrast to Zn(II)-protoporphyrin, protoporphyrin, and DPI (50 μM), none of the heme enzyme inhibitors listed in Table IV inhibited the cytochrome c reductase (CYPOR) activity of microsomes (data not shown).

The results demonstrated NO metabolism by enzymes localized to the endoplasmic reticulum of Caco-2 cells. The respective apparent KM values for O₂, NO and NADPH were similar for microsomes and intact cells: KM(O₂)=9 vs. 17 μM, KM(NO)=300 vs. 200 nM, and KM(NADPH)=2 μM vs. 0.8 μM. The microsomal activity also showed a preference for NADPH over NADH as did digitonin-permeabilized cells. Cyanide inhibited the activity with an IC50 of about 9 μM vs. about 2 μM in cells, and the activity showed comparable CO sensitivity with about 75% inhibition observed at CO:O₂ ratios of 0.4 and 0.75 for cells and microsomes, respectively (FIGS. 11 and 13). Cellular and microsomal NO metabolism also showed comparable sensitivities to ketoconazole, miconazole, econazole, quercetin, garlic extract, Zn(II)-protoporphyrin, and DPI. While not wishing to be bound to a particular theory, differences in substrate saturation and inhibitor sensitivities may be due to different assay conditions or alterations in the activity during isolation. Furthermore, the >2-fold enrichment of NO metabolic activity in microsomal membranes (Table II), and the capacity of anti-CYPOR IgG to inhibit the activity in cell membrane fractions (FIG. 15), demonstrated preferential localization of the NO metabolic activity to the endoplasmic reticulum of cells. The results also demonstrated the role of CYPOR in cellular NO metabolism.

While not bound by a particular theory, the substrate and inhibitor profiles for NO dioxygenation by microsomes and cells also suggest a mechanism similar to that of the NODs (flavohemoglobins) in microbes. In the flavohemoglobin-catalyzed mechanism, the flavin-containing reductase domain transfers electrons from NAD(P)H to the heme-Fe³⁺ in the globin domain to form heme-Fe²⁺. Heme-Fe²⁺ binds O₂ avidly, the stable heme-Fe³⁺(O₂—) complex reacts with NO to form nitrate and heme-Fe³⁺, and the catalytic cycle is re-initiated following heme reduction. The microbial NOD is inhibited by DPI, imidazoles, cyanide, and CO. By analogy, Caco-2 cells appear to utilize the microsomal membrane-bound DPI-sensitive flavin-containing NADPH-dependent CYPOR for electron transfer and an unidentified membrane-bound cyanide and CO-sensitive heme enzyme for NO metabolism. The microsomal NADH:cytochrome b5 oxidoreductase system may account, at least in part, for the residual activity seen with NADH in permeabilized cells and microsomes.

The inventive compositions may be administered to a mammal, such as a human, either prophylactically or in response to a specific condition or disease. The composition may be administered non-systemically such as by topical application, inhalation, aerosol, drops, etc.; systemically by an enteral or parenteral route, including but not limited to intravenous injection, subcutaneous injection, intramuscular injection, intraperitoneal injection, oral administration in a solid or liquid form (tablets (chewable, dissolvable, etc.), capsules (hard or soft gel), pills, syrups, elixirs, emulsions, suspensions, etc.).

As known to one skilled in the art, the composition may contain excipients, including but not limited to pharmaceutically acceptable buffers, emulsifiers, surfactants, electrolytes such as sodium chloride; enteral formulations may contain thixotropic agents, flavoring agents, and other ingredients for enhancing organoleptic qualities.

Different routes of administration and dosing intervals may be used. As examples, a topical application may be applied as needed or at defined intervals; intravenous administration may be continuous or non-continuous; injections may be administered at convenient intervals such as daily, weekly, monthly, etc.; enteral formulations may be administered once a day, twice a day, etc. Instructions for administration may be according to a defined dosing schedule, or an “as needed” basis. The duration and timing of treatment intervals and concentration in the composition can vary. Variables include the extent and type of pathology, how long it takes for the condition to be treated, physician and patient preference, patient compliance, etc.

Any type of suitable, physiologically acceptable topical formulation may be used, as known to one of skill in the art. Examples of such formulations include, but are not limited to, creams, ointments, lotions, emulsions, foams, aerosols, liniments, gels, solutions, suspensions, pastes, sticks, sprays, or soaps. Additionally, the inventive composition may be formulated so that it is encapsulated within a bead, sphere, capsule, microbead, microsphere, microcapsule, liposome, etc., as is known to one skilled in the art. Such formulations may advantageously release the composition over a period of time (time release formulations). The encapsulated formulation may also be prepared as a concentrate or in a dry state or in a powder-like consistency. Such formulations are diluted or reconstituted prior to administration and can be prepared using methods known to one skilled in the art.

The inhibitor-containing composition may also contain other compounds that have desirable therapeutic, cosmetic, and/or aesthetic properties. These may be used in any of the formulations that contain the inhibitor(s). As non-limiting examples, gels or liquids may be useful in some instances in which rapid penetration is desired, such as when treatment occurs at certain intervals or in treatment of pediatric populations. A moisturizing cream base may be useful in other applications, such as in the treatment of geriatric populations.

In the method, a topical formulation of the composition may be applied at or adjacent to the affected site or sites. To limit the exposure to affected skin and to protect unaffected skin, or skin in which treatment is not desired, the composition may be formulated in a viscous material to form an ointment or other formulation in which inadvertent spread is prevented. Skin may also be protected from the composition through the use of physical barriers such as plastic wrap, petrolatum, petroleum jelly, etc. The composition may be formulated in a foam or gel, or within a device which could be cut precisely to the shape of the lesion. Alternatively, the composition may be applied at or adjacent to sites not yet affected, but sought to be treated for preventative or other reasons. The application may be performed in any manner that is suitable to the individual and/or the type of composition, and may additionally involve an application device. The composition may be applied directly or indirectly, such as by a dressing, bandage, covering, etc.

Other variations or embodiments of the invention will also be apparent to one of ordinary skill in the art from the above figures and descriptions. For example, an antimicrobial composition may also include peroxides such as hydrogen peroxide and/or benzoyl peroxide, hypochlorous acid, lysozyme, or other compounds that may provide an additional effect. Thus, the forgoing embodiments are not to be construed as limiting the scope of this invention.

What is claimed is:

1. A biocompatible composition comprising an inhibitor of nitric oxide dioxygenase (NOD) in an amount sufficient to increase the intracellular concentration of nitric oxide (NO) to exert at least one of an antimicrobial, an antineoplastic, or a vasorelaxant effect, and at least one pharmaceutically acceptable excipient.

2. The composition of claim 1 wherein the inhibition is at least one of mammalian NOD or microbial NOD.

3. The composition of claim 1 wherein the inhibitor is at least one of an azole, allicin, quercetin, carbon monoxide, or cyanide.

4. An antimicrobial composition comprising an inhibitor of microbial nitric oxide dioxygenase (NOD) in an amount sufficient to accumulate a toxic concentration of nitric oxide (NO) in the microbe to exert an antimicrobial effect and at least one pharmaceutically acceptable excipient.

5. The composition of claim 4 further comprising at least one of hydrogen peroxide, an organic peroxide, hypochlorous acid, or lysozyme.

6. The composition of claim 4 wherein the inhibitor is at least one of miconazole, econazole, clotrimazole, ketoconazole, or metronidazole.

7. The composition of claim 4 in a formulation for topical administration.

8. An antimicrobial composition comprising a subtoxic amount of nitric oxide (NO) and an amount of an azole sufficient to synergistically mediate NO-induced microbial toxicity.

9. A composition comprising at least one heme-binding compound in an amount effective to inhibit nitric oxide dioxygenase (NOD) and at least one pharmaceutically acceptable excipient.

10. The composition of claim 9 wherein the heme-binding compound is an azole.

11. The composition of claim 9 wherein the heme-binding compound is at least one of miconazole, econazole, ketoconazole, or clotrimazole.

12. The composition of claim 9 wherein the compound inhibits microbial NOD.

13. The composition of claim 9 wherein the compound inhibits mammalian NOD.

14. The composition of claim 9 wherein the compound bonds with at least one hydrophobic group substituent in the conserved hydrophobic distal heme pocket of NOD.

15. A method of reducing microbial growth and activity comprising providing an azole to trap a Fe³⁺ intermediate in nitric oxide dioxygenase catalysis of nitric oxide to nitrate, thereby exerting a microbicidal effect by reducing nitric oxide detoxification.

16. A method of reducing microbial growth and activity comprising accumulating a microbially toxic amount of nitric oxide (NO) by providing an azole thereby inhibiting microbial nitric oxide dioxygenase (NOD).

17. A method of reducing microbial growth and activity comprising inhibiting nitric oxide dioxygenase (NOD)-mediated detoxification of nitric oxide (NO) to nitrate in a microbial cell by providing at least one azole in an inhibitory amount.

18. The method of claim 17 wherein the inhibitory amount of azole ranges from about 1 nM to about 100 μM.

19. A method to decrease microbial antibiotic resistance comprising providing a sub-therapeutic concentration of an antibiotic and an amount of an azole sufficient to inhibit microbial nitric oxide dioxygenase to provide an antimicrobial effect.

20. A method of enhancing microbicidal activity of a nitric oxide antimicrobial comprising providing a subtoxic amount of nitric oxide and an amount of an azole sufficient to synergistically effect nitric oxide toxicity.

21. The method of claim 20 causing at least a two-fold synergy.

22. A method of inhibiting microbial growth and activity comprising providing to a microbe an azole in an amount sufficient to ligand with ferric heme in microbial nitric oxide dioxygenase (NOD) and result in a toxic accumulation of nitric oxide to inhibit microbial growth and activity.

23. A method of inhibiting microbial growth and activity comprising providing an azole inhibitor of nitric oxide dioxygenase (NOD) non-competitive with dioxygen and nitric oxide in inhibiting NOD catalysis.

24. A method for inhibiting microbial nitric oxide dioxygenase (NOD) comprising providing at least one of miconazole, econazole, clotrimazole, ketoconazole, or metronidazole to a organism under conditions sufficient to inhibit microbial NOD.

25. A method of enhancing nitric oxide (NO) toxicity comprising providing nitric oxide and an inhibitor of nitric oxide diooxygenase (NOD) under conditions sufficient to reduce NOD-catalyzed detoxification of toxic NO to nitrate.

26. The method of claim 25 wherein the inhibitor is at least one azole.

27. A method of modulating a therapeutic effect in a mammal comprising providing to the mammal at least one inhibitor of mammalian nitric oxide dioxygenase (NOD) in an amount sufficient to accumulate a concentration of nitric oxide (NO) to modulate at least one of an antineoplastic effect or a vasorelaxant effect.

28. The method of claim 27 wherein tissue NO levels are modulated in response to a steady state oxygen concentration in the tissue.

29. The method of claim 27 wherein the inhibitor increases NO signaling.

30. The method of claim 27 wherein the inhibitor is selected from at least one of an azole, allicin, quercetin, carbon monoxide, or cyanide.
